(19) United States
(12) Patent Application Publication
(10) Pub. No.: US 2023/0190916 A1
(43) Pub. Date: Jun. 22, 2023

(54) COMPOSITIONS AND METHODS FOR DETECTING AND TREATING A SARS-COV-2 INFECTION

(71) Applicants: METACLIPSE THERAPEUTICS CORPORATION, Atlanta, GA (US); EMORY UNIVERSITY, Atlanta, GA (US)

(72) Inventors: Periasamy SELVARAJ, Lilburn, GA (US); Christopher PACK, Atlanta, GA (US); Sampath RAMACHANDIRAN, Duluth, GA (US); J.C. Shaker REDDY, Roswell, GA (US); Luis Enrique MUNOZ, Atlanta, GA (US); Ramireddy BOMMIREDDY, Atlanta, GA (US)

(21) Appl. No.: 17/925,179

(22) PCT Filed: May 14, 2021
(86) PCT No.: PCT/US2021/032541

§ 371 Date: Nov. 14, 2022

Related U.S. Application Data
(60) Provisional application No. 63/024,615, filed on May 14, 2020.

(51) Int. Cl. A61K 39/215; C07K 14/005; C07K 14/535; A61K 47/69; C07K 14/54; G01N 33/569

(52) U.S. Cl. CPC A61K 39/215; A61K 47/6901; C07K 14/005; C07K 14/535; C07K 14/5434; G01N 33/56983; C07K 2319/035; C07K 2319/75; C12N 2770/20023; C12N 2770/20034; C12N 2770/20071; G01N 2333/165; G01N 2469/20

(57) ABSTRACT

Provided herein are recombinant polypeptides comprising a SARS-CoV-2 S1 protein binding domain polypeptide and a GM-CSF polypeptide, polynucleotide sequences encoding the same, virus-like particles comprising the same, and methods for using these compositions for the treatment of a SARS-CoV-2 infection in a subject, and for detection of a SARS-CoV-2 antibodies in a subject.

### CROSS-REFERENCE TO RELATED APPLICATIONS

This application is a national phase entry under 35 U.S.C. §371 of International Application No. PCT/US2021/032541, filed May 14, 2021, which claims the benefit of U.S. Provisional Application No. 63/024,615, filed May 14, 2020, which are expressly incorporated herein by reference in their entireties.

### STATEMENT REGARDING FEDERALLY SPONSORED RESEARCH OR DEVELOPMENT

This invention was made with government support under CA202763 and CA221559 awarded by the National Institutes of Health. The government has certain rights in the invention.

### BACKGROUND

A new strain of coronavirus first appeared in late 2019 in China before beginning its rapid spread across the globe [1]. The disease, named COVID-19, continues to cause severe pneumonia-like symptoms in many of those infected. In addition, it presents a much more severe disease course and fatality rate in the elderly. Coronaviruses, so named for their “crown-like” appearance, are a large family of viruses that spread from animals to humans and include diseases such as Middle East Respiratory Syndrome (MERS) and SARS in addition to COVID-19. While much remains unknown about the new coronavirus, it is known that the virus can spread via human-to-human transmission before any symptoms appear. The virus spike (S) protein binds to the human ACE2 protein for entry [1, 2]. This S protein, and specifically the conserved ACE-2 receptor binding domain (RBD), are potential targets for vaccine design. Antibodies directed to the S protein and the RBD have been shown to be neutralizing using in vitro assays for coronaviruses [3], suggesting that an effective vaccine can be produced to prevent SARS-CoV-2 infection.

Although current vaccines are effective in young adults, the elderly fail to respond well to many vaccines. Due to immunosenescence, which affects both innate and adaptive immunity, the elderly have decreased immune responses to vaccines [13, 14]. For example, 250,000 to 500,000 older patients aged over 65 years die annually because of influenza-related complications [15]. Since SARS-CoV-2 virus causes mortality disproportionately in the elderly, developing vaccines with better efficacy in aged population is an urgent public health need. Development of new adjuvants or combination of existing adjuvants that enhance the immunogenicity while maintaining an acceptable safety profile is one of the current strategies to improve the vaccine efficacy in elderly population.

There is a growing repertoire of substances being investigated as adjuvants. However, only a few adjuvants such as Aluminum salts (Alum), Monophosphoryl lipid A (MPL), MF59 (oil in water emulsion) and virosomes are approved by the FDA for human use [16]. Among the approved adjuvants, MF59 (biodegradable squalene in water emulsion) was developed specifically to address the low efficacy of influenza vaccine in elderly [17]. Fluad influenza vaccine (Novartis) containing MF59 as an adjuvant was shown to be 25% more effective compared to unadjuvanted trivalent inactivated influenza vaccine (TIV) in elderly patients [18, 19]. Although several phase III clinical trials involving aged patients using MPL or ASO3 (oil in water emulsion) were promising, the improvement was only incremental [15, 20, 21]. In addition, many TLR agonists have shown to be less effective in the elderly than in the young, primarily due to reduced signaling capacity and decreased TLR receptor expression in aged antigen presenting cells [22]. This is particularly relevant for TLR9 and CpG as an adjuvant for influenza viral vaccines [23]. Also, phagocytic and antigen cross-priming capacity in aging dendritic cells is impaired [24]. In addition, targeting of antigen to APC via GM-CSF receptor may enhance cross-priming. Adjuvants that have a tolerable safety profile and generate a Th1 immune response are of high importance. Alum, the first compound approved as an adjuvant, elicits a Th2 immune response rather than Th1, and is ineffective in inducing a strong cytotoxic-T lymphocyte (CTL) response [25, 26]. Several adjuvants (Alum, MPL, CpG, Poly I:C, and cholera toxin) have been investigated to enhance influenza VLP vaccines, but none have been commercially developed for use [27].

A number of studies documented the mechanism by which age dependent decline in immune response modulates functions of immune cells qualitatively and quantitatively. These studies also suggest some strategies to circumvent and improve the immune response in aged individuals [28, 29]. Aged mice produce lower levels of IgG in response to influenza infection, which could be the consequence of a compromised CD4 T helper response [29]. Cytokine combinations including TNF-α, IL-1β and IL-6 have been shown to improve CD4 T cell responses to provide help to B cells in aged mice. It was also shown that IL-6 dependent activation of dendritic cells from aged mice mitigates the age- associated decline in the initiation of immune response and survival of aged T cells [29]. Moreover, proinflammatory cytokines at the dendritic cell/T cell junction has been shown to play an important role in inducing a robust immune response [29].

It is well documented that cytokines increase the efficacy of vaccines by attracting and activating key immune cells. Two such cytokines being evaluated for their potential as adjuvants are Interleukin-12 (IL-12) and granulocyte macrophage colony-stimulating factor (GM-CSF). IL-12, a heterodimeric cytokine (p35 and p40 subunits), activates dendritic cells (DCs), T lymphocytes and natural killer (NK) cells to release IFN-y, TNF□ etc. [30-32]. IL-12 also sends a strong signal to T-helper cell precursors to differentiate towards a Th1 lineage, which also promotes development of a robust CTL response [33]. Pre-clinical and clinical trials performed to evaluate the potential of recombinant soluble IL-12 as an adjuvant in treating several cancers and viral hepatitis resulted in enhanced immune response [34-38], but also resulted in unfavorable side effects and systemic toxicity [39, 40]. However, membrane anchoring of IL-12 has been shown to prevent these unwanted side effects in a comprehensive toxicology studies in mice performed by Metaclipse even at 5X higher biologically active dose.

GM-CSF potentiates a strong immune response primarily through maturation and differentiation of dendritic cells [41-44]. Blood monocytes derived from young (<30 years) and old (> 65 years) people differentiate to DCs in response to GM-CSF and IL-4 and produce similar amounts of inflammatory cytokines like IL-12 and TNF-alpha when stimulated with whole inactivated influenza virus [45] suggesting that aged DCs are as effective as young when induced by cytokines like GM-CSF and IL-4. The FDA approved a prostate cancer vaccine, Provenge®, by Dendreon, uses a single antigen fused to GM-CSF and shown to effectively deliver antigen to the immune system [46, 47]. In several cancer vaccine development studies, co-administration of GM-CSF with either single or multiple peptide antigens showed cytotoxic T cell immune responses relative to vaccine alone [48-51]. Although many adjuvants including MF59 were effective in improving the influenza vaccine efficacy, they are known to induce several side effects because of reactogenicity and toxicity [29].

### DESCRIPTION OF DRAWINGS

FIG. 1. Schematic showing creation of a virus-like particle (VLP) comprising a GPI-recombinant polypeptide comprising a SARS-CoV-2 S1 protein receptor binding domain (RBD) polypeptide and a GM-CSF polypeptide anchored to the VLP and a GPI- recombinant polypeptide comprising IL-12 anchored to the VLP.

FIGS. 2A-2B. Virus-specific serum IgG antibody responses after immunization of young adult and aged mice. (A) Virus specific IgG antibodies in young adult mice. (B) Virus specific IgG antibodies in aged mice. Young adult (6 weeks) BALB/c mice or aged (18 months) BALB/c mice (n=5 per group) were prime and boost immunized with HA VLP (1 µg) or HA VLP (1 µg) containing cytokines (GPI-GM-CSF and IL-12). At 3 weeks post immunization serum IgG antibody levels were determined by ELISA using homologous virus (inactivated A/PR8 virus) antigens.

FIGS. 3A-3B. VLP-cytokine based vaccines provide enhanced cross protection in young adult mice. Body weight changes in young adult mice (BALB/c) after challenge with homologous A/PR8/34 H1N1 virus (A) and heterologous A/WSN/33 H1N1 virus (B). At 3 weeks after boost immunization, naïve and vaccinated young adult mice (n=5) were challenged, and body weight changes were monitored. data. Naïve inf: Unvaccinated mice after infection with virus, HA-VLP: VLPs containing HA only, HA-VLP-Cyt: HA - VLP incorporated with GPI-anchored GM-CSF and IL-12.

FIGS. 4A-4C. Cross protective efficacy in aged mice after vaccination with HA VLPs incorporated with GPI-GM-CSF and GPI-IL-12. (A) Body weight changes in aged mice after heterologous challenge with antigenically different A/WSN virus. (B) Lung virus titers at 7 days after challenge were determined by egg inoculation assays. (C) Spleen cells collected from vaccinated mice at 7 days after challenge were cultured for 3 days with inactivated A/PR8 (iPR8) or inactivated A/WSN (iWSN) virus as a stimulator. Levels of IFN-gamma secreting cells (per 10^6) were measured by counts of spots using DAB solution.

FIGS. 5A-5E. Serum IgG and IgG antibody secreting cell responses in aged mice after heterologous challenge. (A) Serum IgG1, (B) IgG1 isotype, (C) IgG2a isotype antibody responses specific for A/PR8 virus in aged mice day 7 post challenge with A/WSN virus. (D-E) In vitro IgG antibody producing cell responses in draining lymph nodes collected day 7 post challenge with A/WSN virus. A/PR8 specific IgG antibody levels were determined by ELISA in culture supernatants after 1 day or 5 days cultures.

FIGS. 6A-6I. Design, expression, and characterization of GM-CSF-GPI-S 1 RBD fusion protein. (A) Structure of SARS-CoV-2 and Spike (S) protein domains, (B) Design of GM-CSF-GPI-S1 RBD fusion protein gene. (C) Detection of murine GM-CSF-GPI-S 1 RBD fusion protein on CHO-S cell transfectants by flow cytometry, (D) Human ACE-2 binding to GPI-RBD-GM-CSF fusion protein CHO S cells as detected by flow cytometry. (E) Colloidal blue (lanes 1 & 2), and (F & G) Western blot (lanes 3-10) of the purified fusion protein from CHO-S cells (lanes 4 & 5 in F, 8 & 9 in G) probed with anti-RBD antibody (lanes 4 & 5 in F), or anti-GM-CSF mAb (lanes 8 & 9 in G). Lane 3 is control RBD probed with anti-RBD Ab, and lane 7 is control GM-CSF probed with anti-GM-CSF antibody. (H) ELISA using purified GPI-RBD-GM-CSF fusion protein. Streptavidin-FITC was used to detect biotinylated human ACE-2 binding in D and Streptavidin-HRP in ELISA in H. (I) Influenza VLP with GPI-IL-12 and GPI-RBD-GM-CSF fusion protein determined by flow cytometery.

FIG. 7. Schematic showing various GPI-recombinant polypeptides included in the present invention.

FIGS. 8A-8B. Purified RBD-GM-CSF fusion protein binds to antibodies from convalescent sera (A) and mouse GM-CSF in the fusion protein retains functional capacity to induce BMDC proliferation (B). (A) ELISA plates were coated with GPI-RBD-GM-CSF fusion protein and binding of antibodies in human convalescent plasma (Ray Biotech Inc) were determined using HRP-conjugated anti-human IgG and TMB substrate. (B) mouse bone marrow derived dendritic cells (BMDC) were cultured in vitro with GPI-RBD-GM-CSF and the proliferation was determined using XTT assay in a 96-well microtiter plate.

FIGS. 9A-9B. VLP vaccine induces antibody against GPI-RBD-GM-CSF. ELISA plates coated with GPI-RBD-GM-CSF (3 µg/ml) overnight at 4° C. and then blocked with assay diluent, incubated with diluted serum samples and developed with anti-mouse IgG-HRP (A) or HRP-conjugated isotype specific antibodies (B). Background OD is 0.05.

FIG. 10. VLP vaccine induced antibody recognizes S1 RBD. VLP vaccine induced antibody in the mouse sera (1:5000 dilution, 6 months) binds to Spike S1 RBD as determined by ELISA.

FIG. 11. VLP vaccine induces effector T cell cytokine response: Splenocytes prepared after 10 days of immunization, stimulated with Spike S1 RBD peptide pools for 12 hrs, (1x10⁶ cells per well in 96 well plate) in the presence of anti-CD28 Ab and Brefeldin A was added for 5 hours, total 17 hrs culture. Cells were stained for surface markers and then for intracellular cytokines according to the staining protocol from BioLegend. Cells were acquired on a CYTEK Aurora flow cytometer and data analyzed by FlowJo software.

FIGS. 12A-12C. VLP vaccine-induced SARS-CoV-2 virus neutralizing antibodies. CHO S cells expressing GPI-RBD-GM-CSF were used for ACE2 binding studies for detecting neutralizing antibodies in the mouse sera from VLP vaccinated mice. Cells were incubated with diluted plasma (1:10 dilution) from week 4 (A) or week 8 (B) and then biotinylated ACE-2 was added to the cells and detected by Streptavidin-FITC by flow cytometry. (C) Plasma from vaccinated mice (week 12) inhibits WA1 strain of SARS-CoV-2 infection of Vero E6 cells in vitro

FIG. 13. VLP vaccine-induced antibody recognizes South African variant S1 RBD. VLP vaccine induced antibody in the mouse sera binds to South African (SA) variant of Spike S1 and original spike S1 protein as determined by ELISA.

FIG. 14. GPI-RBD-human GM-CSF binds to ACE2. ELISA plates coated with GPI-RBD-human GM-CSF fusion protein (3 µg/ml) and then detected with biotinylated ACE-2 (0.02, 0.2 and 2.0 µg/ml) and streptavidin-HRP (1:500 dilution). MM57 monoclonal antibody (Sino Biological) was used as positive control which is specific to Spike S1 RBD with demonstrated neutralizing activity. Anti-mouse IgG-HRP (1:4000 dilution) was used for detecting MM57 mAb.

FIGS. 15A-15B. VLP vaccine induced antibody recognizes S1 RBD and influenza antigen(s). VLP vaccine induced antibody in the mouse sera (1:5000 dilution, 10 weeks after booster dose) binds to Spike S1 RBD (A) and influenza VLP (B) as determined by ELISA.

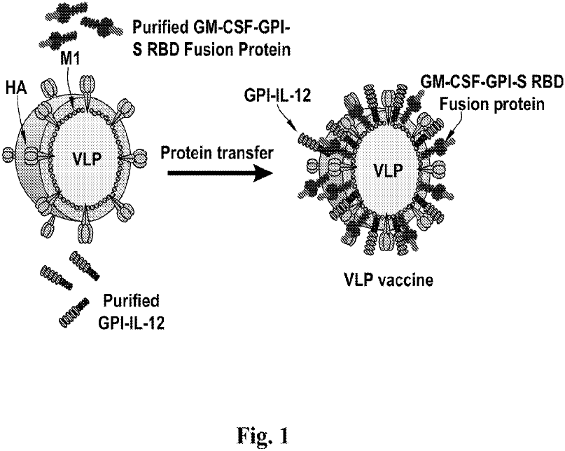

### SUMMARY

Provided herein are isolated recombinant polypeptides comprising a SARS-CoV-2 S1 protein binding domain polypeptide and a GM-CSF polypeptide. In some embodiments, the recombinant polypeptide of claim 1, SARS-CoV-2 S1 protein binding domain polypeptide comprises a full-length S1 polypeptide. In some embodiments, the SARS-CoV-2 S1 protein binding domain polypeptide comprises SEQ ID NO:1 or a sequence at least 80% identical to SEQ ID NO:1. In some embodiments, the SARS-CoV-2 S1 protein binding domain polypeptide comprises SEQ ID NO: 6 or SEQ ID NO:7. In some aspects, the GM-CSF polypeptide comprises SEQ ID NO:2 or is at least 80% identical to SEQ ID NO:2.

Also provided herein are isolated recombinant polynucleotides comprising a SARS-CoV-2 S1 protein receptor binding domain (RBD) polynucleotide, a GM-CSF polynucleotide and a GPI-anchoring signal polynucleotide. In some embodiments, the SARS-CoV-2 S1 proteinreceptor binding domain polynucleotide and the GM-CSF polynucleotide are operably linked. In some embodiments, the SARS-CoV-2 S1 protein receptor binding domain polynucleotide comprises a full-length S1 polynucleotide. In some embodiments, the isolated recombinant polynucleotides comprising a SARS-CoV-2 S1 protein receptor binding domain (RBD) polynucleotide, a GM-CSF polynucleotide and a GPI-anchoring signal polynucleotide comprise SEQ ID NO:12. In some embodiments, the isolated recombinant polynucleotides comprising a SARS-CoV-2 S1 protein receptor binding domain (RBD) polynucleotide, a GM-CSF polynucleotide and a GPI-anchoring signal polynucleotide comprise SEQ ID NO:14.

Included herein are methods of detecting an antibody specific for a SARS-CoV-2 S1 protein receptor binding domain in a subject comprising, obtaining a blood sample from the subject, contacting the sample with a recombinant polypeptide comprising a SARS-CoV-2 S1 protein receptor binding domain polypeptide and a GM-CSF polypeptide, and detecting specific binding between the recombinant polypeptide and an antibody in the blood sample, wherein detection of specific binding indicates a presence of the antibody specific for a SARS-CoV-2 S1 protein receptor binding domain in the subject. In some aspects, the method of detection further comprises treating the subject for a SARS-CoV-2 infection. In some aspects, the subject is a human.

Also included herein are enveloped virus-like particles (VLPs) comprising a GPI-recombinant polypeptide anchored to the VLP, wherein the GPI-recombinant polypeptide comprises a SARS-CoV-2 S1 protein receptor binding domain polypeptide and a GM-CSF polypeptide (GPI-RBD-GM-CSF polypeptide). In some aspects, the VLP further comprises one or more GPI-immunostimulatory molecules anchored to the VLP. In some aspects, the one or more GPI-immunostimulatory molecules is a GPI-IL-12 molecule. In some aspects, the VLP is derived from an influenza virus, including, but not limited to, an influenza virus A/PR8. In some VLP embodiments, the SARS-CoV-2 S1 protein receptor binding domain polypeptide comprises SEQ ID NO:1 or comprises a sequence at least 80% identical to SEQ ID NO:1. In some VLP embodiments, the SARS-CoV2 S1 protein receptor binding domain polypeptide comprises SEQ ID NO:6 or SEQ ID NO:7, or comprises a sequence at least 80% identical to SEQ ID NO:6 or SEQ ID NO:7. In some VLP embodiments, the GM-CSF polypeptide comprises SEQ ID NO:2 or is at least 80% identical to SEQ ID NO:2.

Further included herein are methods for reducing a SARS-CoV-2 infection in a subject, comprising administering to the subject a therapeutically effective amount of an enveloped virus-like particle (VLP) described herein, wherein the infection in the subject is reduced as compared to a control. In some embodiments, the VLP further comprises one or more GPI-immunostimulatory molecules anchored to the VLP. In some embodiments, the GPI-immunostimulatory molecules is a GPI-IL-12 molecule.

### DETAILED DESCRIPTION

Provided herein are compositions and methods for preventing or decreasing the severity of a SARS-CoV-2 infection in a subject and for detecting a SARS-CoV-2 infection in a subject. Included herein are recombinant polynucleotides comprising a SARS-CoV-2 S1 protein binding domain polynucleotide and a GM-CSF polynucleotide, recombinant polypeptides comprising a SARS-CoV-2 S1 protein binding domain polypeptide (RBD) and a GM-CSF polypeptide and virus-like particles that comprise the recombinant RBD-GM-CSF polypeptides.

### Terminology

Unless defined otherwise, all technical and scientific terms used herein have the same meaning as commonly understood to one of ordinary skill in the art to which this invention belongs. The following definitions are provided for the full understanding of terms used in this specification.

As used in the specification and claims, the singular form “a,” “an,” and “the” include plural references unless the context clearly dictates otherwise. For example, the term “an agent” includes a plurality of agents, including mixtures thereof.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value” 10” is disclosed, then “about 10” is also disclosed.

“Administration” or “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject’s body (e.g. greater than 50% of the body), for example through entrance into the circulatory or lymphatic systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject’s body. Administration includes self-administration and the administration by another.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

The term “derived from” refers to a VLP that has been obtained from a virus. Methods of obtaining VLPs from viruses are well known to those of skill in the art and include the methods described herein and in U.S. Pat. No. 10,729,760.

The term “identity” “identical to” or “homology” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. Neither N- nor C-terminal extensions nor insertions shall be construed as reducing identity or homology. A polynucleotide or polynucleotide region (or a polypeptide or polypeptide region) that has a certain percentage (for example, 80%, 85%, 90%, or 95%) of “sequence identity” to another sequence means that, when aligned over their full lengths, that percentage of bases (or amino acids) are the same in comparing the two sequences. This alignment and the percent homology or sequence identity can be determined using software programs known in the art. In one embodiment, default parameters are used for alignment. In one embodiment a BLAST program is used with default parameters. In one embodiment, BLAST programs BLASTN and BLASTP are used with the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Pharmaceutically acceptable” component can refer to a component that is not biologically or otherwise undesirable, e.g., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

The terms “polynucleotide” and “oligonucleotide” are used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

“Polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics.

The terms “specific binding,” “specifically binds,” “selective binding,” and “selectively binds” mean that a polypeptide such as an antibody exhibits appreciable affinity for a particular binding partner polypeptide such as a SARS-CoV-2 S1 protein receptor binding domain polypeptide. Appreciable binding affinity includes binding with an affinity of at least 10⁶ M⁻¹, specifically at least 10⁷ M⁻¹, more specifically at least 10⁸ M⁻¹, yet more specifically at least 10⁹ M⁻¹, or even yet more specifically at least 10¹⁰ M⁻¹. A binding affinity can also be indicated as a range of affinities, for example, 10⁶ M⁻¹ to 10¹⁰ M⁻¹, specifically 10⁷ M⁻¹ to 10¹⁰ M⁻¹, more specifically 10⁸ M⁻¹to 10¹⁰ M⁻¹. Specific binding can be determined according to any art-recognized means for determining such binding. In some embodiments, specific binding is determined according to Scatchard analysis and/or competitive binding assays.

In some embodiments, “specific binding,” “specifically binds,” “selective binding,” and “selectively binds” when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. In some embodiments, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

“Therapeutically effective amount” or “therapeutically effective dose” of a composition (e.g. a composition comprising an agent) refers to an amount that is effective to achieve a desired therapeutic result. In some embodiments a desired therapeutic result is prevention of a SARS-CoV-2 viral infection or a reduction in the severity of a SARS-CoV-2 viral infection. In some embodiments, a desired therapeutic result is a reduction in an amount of detectable SARS-CoV-2 virus or antigen in a subject. Therapeutically effective amounts of a given agent will typically vary with respect to factors such as the type and severity of the disorder or disease being treated and the age, gender, weight, and general condition of the subject. Thus, it is not always possible to specify a quantified “therapeutically effective amount.” However, an appropriate “therapeutically effective amount” in any subject case may be determined by one of ordinary skill in the art using routine experimentation. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. It is understood that, unless specifically stated otherwise, a “therapeutically effective amount” of a therapeutic agent can also refer to an amount that is a prophylactically effective amount. In some instances, a desired biological or medical response is achieved following administration of multiple dosages of the composition to the subject over a period of days, weeks, or years.

“Treat,” “treating,” “treatment,” and grammatical variations thereof as used herein, include the administration of a composition with the intent or purpose of partially or completely, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease. In some embodiments, “treating” refers to prevention of a SARS-CoV-2 viral infection or a reduction in the severity of a SARS-CoV-2 viral infection in a subject as compared with prior to treatment of the subject or as compared with the incidence of such symptom in a general or study population. In some embodiments, “treating” refers to a reduction in an amount of detectable SARS-CoV-2 virus or antigen in a subject as compared with the incidence of such symptom in a general or study population.

As used herein the term “virus-like particle” is used interchangeably with “VLP” and refers to a structure that in at least one attribute resembles a virus, but which has not been demonstrated to be infectious. Virus-like particles in accordance with the invention lack a viral genome. In some embodiments, the virus-like particles comprise viral structural proteins. In some embodiments, the virus-like particles are enveloped, whereas in other embodiments, the virus-like particles are non-enveloped. Enveloped VLPs comprise a lipid bilayer. In some embodiments, the virus-like particles are derived from an influenza virus. The influenza virus VLPs can be derived from any of influenza virus A, influenza virus B, influenza virus C and influenza virus D. In some embodiments, the influenza virus VLPs are derived from influenza virus A/PR8 (H1N1).

As used herein, the term “anchored to the VLP” refers to the insertion of an exogenous polypeptide such as GM-CSF, SARS CoV-2 S1 subunit receptor binding domain (RBD) and/or IL-12 at the exterior of the VLP surface. The term “anchored to the VLP” does not refer to endogenous polypeptides naturally expressed at a cell’s surface before viral budding from the cell. In some embodiments, the exogenous polypeptide is anchored to the lipid bilayer of the VLP via a glycosyl-phosphatidyl-inositol (GPI) molecule. In some embodiments, the GPI-anchor comprises a glycosylated moiety attached to phosphatidylinositol containing two fatty acids. In some embodiments, a GPI molecule is composed of a phosphatidylinositol group linked through a carbohydrate-containing linker (such as glucosamine and mannose glycosidically bound to the inositol residue) and an ethanolamine phosphate (EtNP), wherein the EtNP is attached to the C-terminal amino acid of the polypeptide. The two fatty acids within the phosphatidyl-inositol group anchor the polypeptide to the VLP membrane.

### Compositions

As explained above, the present invention includes compositions and methods for preventing or decreasing the severity of a SARS-CoV-2 infection in a subject and for detecting a SARS-CoV-2 infection in a subject. A SARS-CoV-2 infection is commonly referred to as a “coronavirus infection.” Also included herein are recombinant polynucleotides comprising a SARS-CoV-2 S1 protein receptor binding domain (RBD) polynucleotide and a GM-CSF polynucleotide, recombinant polypeptides comprising a SARS-CoV-2 S1 protein receptor binding domain polypeptide (RBD) and a GM-CSF polypeptide, and virus-like particles (VLPs) that comprise the recombinant RBD-GM-CSF polypeptides in a GPI-anchor form. In some embodiments, the VLPs further comprise a GPI-anchored form of one or more immunostimulatory molecules (referred to herein as a GPI-immunostimulatory molecule). As used herein “immunostimulatory molecule” refers to any molecule that, when attached to a VLP, can stimulate or co-stimulate an immune response to a SARS-CoV-2 virus. In some embodiments, the one or more immunostimulatory molecules are cytokines. In some embodiments, the cytokine is an IL-12.

GPI-anchored molecules are incorporated into virus-like particles (VLPs) by a ‘protein transfer’ technique. In this approach, polypeptides are converted to GPI-anchored forms by attaching the DNA sequence corresponding to a cytokine to the DNA sequence corresponding to the GPI-anchor signal sequence from naturally occurring GPI-anchored CD59. In some embodiments, the GPI-anchor comprises a glycosylated moiety attached to phosphatidylinositol containing two fatty acids. The GPI-anchor permits incorporation of GPI-anchored proteins into the lipid bilayer of influenza VLPs [52]. By introducing membrane incorporated GPI-cytokines into VLPs, multiple viral-specific antigens can be presented to the immune system to mount a robust immune response. In addition, administration of VLP vaccines containing membrane-anchored cytokines will localize the cytokines to the area of injection, thereby reducing the toxic effects associated with soluble cytokines.

According to the present invention, the SARS-CoV-2 S1 protein receptor binding domain (RBD) refers to the region of the SARS CoV-2 spike protein that specifically binds to a receptor ACE2 on a host cell. In some embodiments, the RBD polypeptide comprises amino acids 319 through 541 of the spike protein. In some embodiments, the RBD polypeptide comprises SEQ ID NO:1. It should be understood, however, that as used herein, a SARS-CoV-2 S1 protein receptor binding domain polynucleotide or polypeptide refers to any polynucleotide or polypeptide that comprises the region of the SARS CoV-2 spike protein that specifically binds to a receptor ACE2 on a host cell. Accordingly, a SARS-CoV-2 S1 protein receptor binding domain polynucleotide or polypeptide includes a full-length S1 polynucleotide or polypeptide, and any fragment thereof that includes the region of the SARS CoV-2 spike protein that specifically binds to a receptor ACE2 on a host cell. In some embodiments, the SARS-CoV-2 S1 protein receptor binding domain polypeptide comprises SEQ ID NO:6 or SEQ ID NO:7.

The GM-CSF polynucleotides and polypeptides can be human or of another species. In some embodiments, the GM-CSF polynucleotides and polypeptides are murine. In other embodiments, the GM-CSF polynucleotides and polypeptides are human. In some embodiments, the GM-CSF polynucleotide or polypeptide is that found in one or more publicly available databases as follows: HGNC: 2434, Entrez Gene: 1437, Ensembl: ENSG00000164400, OMIM: 138960, and UniProtKB: P04141. In some embodiments, the IL-12 comprises a polypeptide sequence having about 70% or greater, about 75% or greater, about 80% or greater, about 85% or greater, about 90% or greater, about 95% or greater, or about 98% or greater homology or identity with SEQ ID NO:2.

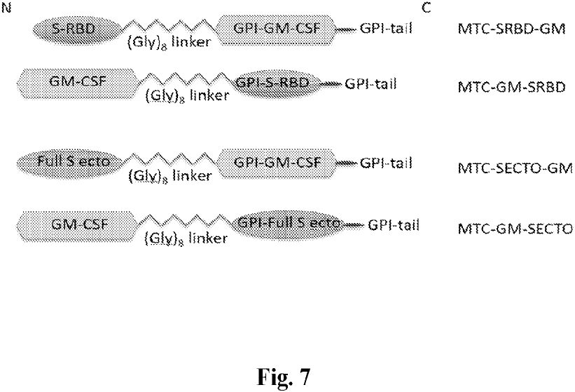

The recombinant polypeptides comprising a RBD polypeptide and a GM-CSF polypeptide can be any combination of the above referenced polypeptides. In some embodiments, the GM-CSF polypeptide is attached to the GPI-anchor. In other embodiments the RBD polypeptide is attached to the GPI-anchor. FIG. 7 shows various examples of such constructs. In some embodiments, the recombinant polypeptide comprising a RBD polypeptide and a GM-CSF polypeptide is encoded by a polynucleotide sequence comprising SEQ ID NO:12 or SEQ ID NO:14. In some embodiments, the recombinant polypeptide comprising a RBD polypeptide and a GM-CSF polypeptide is encoded by a polynucleotide sequence having about 70% or greater, about 75% or greater, about 80% or greater, about 85% or greater, about 90% or greater, about 95% or greater, or about 98% or greater homology or identity with SEQ ID NO:12 or SEQ ID NO:14.

In some embodiments, IL-12 comprises IL-12a and IL-12b. In some embodiments, the IL-12 comprises the sequence of SEQ ID NO:3, or a fragment thereof. In some embodiments, the IL-12 polynucleotide or polypeptide is that found in one or more publicly available databases as follows: HGNC: 5969 Entrez Gene: 3592 Ensembl: ENSG00000168811 OMIM: 161560 UniProtKB: P29459. In some embodiments, the IL-12 comprises a polypeptide sequence having about 70% or greater, about 75% or greater, about 80% or greater, about 85% or greater, about 90% or greater, about 95% or greater, or about 98% or greater homology or identity with SEQ ID NO:3.

In some embodiments, the IL-12 polynucleotide or polypeptide is that found in one or more publicly available databases as follows: HGNC: 5970 Entrez Gene: 3593 Ensembl: ENSG00000113302 OMIM: 161561 UniProtKB: P29460. In some embodiments, the IL-12 comprises a polypeptide sequence having about 70% or greater, about 75% or greater, about 80% or greater, about 85% or greater, about 90% or greater, about 95% or greater, or about 98% or greater homology or identity with SEQ ID NO:4.

As used herein “GPI-recombinant polypeptide” refers to a recombinant polypeptide having a GPI anchor. “GPI-immunostimulatory molecule” refers to an immunostimulatory molecule having a GPI anchor. GPI-anchored polypeptides can be created through the addition of a GPI anchor signal sequence to the polypeptide. A GPI anchor signal sequence is a sequence that directs GPI anchor addition to the polypeptide. One example of a GPI anchor signal sequence that may be added to a polypeptide is SEQ ID NO:5, a CD59 GPI anchor signal sequence. Accordingly, in some embodiments, the immunostimulatory agent, antigen, or other molecules attached to the lipid membrane include a GPI anchor signal sequence.

A number of proteins commonly expressed by cells are attached to the cell membrane via a GPI-anchor. These proteins are post-translationally modified at their carboxy terminus to express this glycosylated moiety which is synthesized in the endoplasmic reticulum. These naturally expressing GPI-anchored molecules are widely distributed in mammalian cells and serve a host of different cellular functions, such as cell adhesion, enzymatic activity, and complement cascade regulation. Naturally occurring GPI- anchored proteins lack a transmembrane and cytoplasmic domain that otherwise anchor membrane proteins. The GPI-anchor consists of a glycosylated moiety attached to phosphatidylinositol containing two fatty acids. The phosphatidylinositol portion, as well as an ethanolamine which is attached to the C-terminal of the extracellular domain of the membrane proteins, anchor the molecule to the cell membrane lipid bilayer.

In order to exploit this natural linkage using recombinant DNA techniques, the transmembrane and cytoplasmic domains of a transmembrane surface protein need only be replaced by the signal sequence for GPI-anchor attachment that is found at the hydrophobic C-terminus of GPI-anchored protein precursors. This method may be used to generate GPI- anchored proteins is not limited to membrane proteins; attaching a GPI-anchor signal sequence to a secretory protein also converts the secretory protein to a GPI-anchored form. The method of incorporating the GPI-anchored proteins onto isolated cell surfaces or TMVs is referred to here as protein transfer.

GPI-anchored molecules can be incorporated onto lipid membranes spontaneously. GPI-anchored proteins can be purified from one cell type and incorporated onto cell membranes of a different cell type. GPI-anchored proteins can be used to customize the lipid membranes disclosed herein. Multiple GPI-anchored molecules can be simultaneously incorporated onto the same cell membrane. The amount of protein attached to the VLP can be controlled by simply varying the concentration of the GPI-anchored molecules to be incorporated onto membranes. A significant advantage of this technology is the reduction of time in preparing vaccines from months to hours. These features make the protein transfer approach a more viable choice for the development of cancer vaccines for clinical settings. The molecules incorporated by means of protein transfer retain their functions associated with the extracellular domain of the native protein.

In some embodiments, the polynucleotide encoding a SARS-CoV-2 S1 protein binding domain polypeptide comprises SEQ ID NO:8 or a polynucleotide sequence having about 70% or greater, about 75% or greater, about 80% or greater, about 85% or greater, about 90% or greater, about 95% or greater, or about 98% or greater homology or identity with SEQ ID NO:8.

In some embodiments, the recombinant polynucleotide encoding a SARS-CoV-2 S1 protein binding domain polypeptide and a GM-CSF polypeptide comprises SEQ ID NO:12 or SEQ ID NO:14. In some embodiments, the recombinant polynucleotide encoding a SARS-CoV-2 S1 protein binding domain polypeptide and a GM-CSF polypeptide comprises has about 70% or greater, about 75% or greater, about 80% or greater, about 85% or greater, about 90% or greater, about 95% or greater, or about 98% or greater homology or identity with SEQ ID NO: 12 or SEQ ID NO:14. In some embodiments, the recombinant polynucleotide encoding a SARS-CoV-2 S1 protein binding domain polypeptide and a GM-CSF polypeptide comprises a GPI-anchoring signal polynucleotide, a S1 protein binding domain polynucleotide and a human GM-CSF polynucleotide of SEQ ID NO: 12. SEQ ID NO: 14 is a fusion polynucleotide consisting of the human GM-CSF and SARS CoV-2 S protein RBD encoding a glycoprotein with a Glycosylphosphatidylinositol anchor. Its molecular weight is approximately 60 kD. The deduced encoded amino acid sequence is identical to the sequence for mouse GM-CSF and SARS-CoV-2 S protein RBD (except for N501Y, E484K, and K417N mutations described in the South Africa mutant SARS-CoV-2 virus) attached to the GPI anchor from human CD59.

In some embodiments, the recombinant polynucleotide encoding a SARS-CoV-2 S1 protein binding domain polypeptide and a GM-CSF polypeptide comprises SEQ ID NO:11 or SEQ ID NO:13. In some embodiments, the recombinant polynucleotide encoding a SARS-CoV-2 S1 protein binding domain polypeptide and a GM-CSF polypeptide comprises has about 70% or greater, about 75% or greater, about 80% or greater, about 85% or greater, about 90% or greater, about 95% or greater, or about 98% or greater homology or identity with SEQ ID NO:11 or SEQ ID NO:13. SEQ ID NO:11 comprises a GPI-anchoring signal polynucleotide, a S1 protein binding domain polynucleotide and a mouse GM-CSF polynucleotide. SEQ ID NO:13 is a fusion polynucleotide consisting of the mouse GM-CSF and SARS CoV-2 S protein RBD encoding a glycoprotein with a Glycosylphosphatidylinositol anchor. Its molecular weight is approximately 60 kD. The deduced encoded amino acid sequence is identical to the sequence for mouse GM-CSF and SARS-CoV-2 S protein RBD (except for N501Y, E484K, and K417N mutations described in the South Africa mutant SARS-CoV-2 virus) as well as the addition of 10 amino acids to the C-terminus) attached to the GPI anchor from human CD59.

### Methods for Detecting and Treating

Disclosed herein is a method for detecting an antibody specific for a SARS-CoV-2 S1 protein binding domain in a subject comprising, obtaining a blood sample from the subject, contacting the sample with a recombinant polypeptide comprising a SARS-CoV-2 S1 protein binding domain polypeptide and a GM-CSF polypeptide, and detecting specific binding between the recombinant polypeptide and an antibody in the blood sample, wherein detection of specific binding indicates a presence of the SARS-CoV-2 S1 protein binding domain in the subject. In some embodiments, the detecting method further comprises treating the subject for a SARS-CoV-2 infection.

One method for treating a SARS-CoV-2 infection in a subject comprises administering to the subject a therapeutically effective amount of an enveloped virus-like particle (VLP) comprising a GPI-recombinant polypeptide comprising a SARS-CoV-2 S1 protein binding domain polypeptide and a GM-CSF polypeptide (GPI-RBD-GM-CSF) anchored to the VLP. In some embodiments, the enveloped VLP further comprises a GPI-IL-12 molecule anchored to the VLP.

For all of the detection and treatment methods, the VLP, the GPI-RBD-GM-CSF and the GPI-IL-12 can be any as described herein.

The subject can be any mammalian subject, for example a human, dog, cow, horse, mouse, rabbit, etc. In some embodiments, the subject is a primate, particularly a human. The subject can be a male or female of any age, race, creed, ethnicity, socio-economic status, or other general classifiers.

In the treatment methods, the dosage forms of the compositions disclosed herein can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavenous, intrathecal, intravitreal, intracerebral, gingival, subgingival, intracerebroventricular, and intradermal. Such formulations may be prepared by any method known in the art.

The disclosed treatment methods can be performed any time prior to and/or after the onset of a disease (e.g., a SARS-CoV-2 infection) or administration of a vaccine. In some aspects, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years;12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of a disease (e.g., SARS-CoV-2 infection) or administration of a vaccine; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of a disease (e.g., SARS-CoV-2 infection) or administration of a vaccine.

Dosing frequency for the compositions disclosed herein, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiment, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiment, the dosing frequency for the T cells disclosed herein includes, but is not limited to, at least once a day, twice a day, or three times a day. In some embodiment, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiment, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiment, the interval between each administration is constant. For example, the administration can be carried out daily, every two days, every three days, every four days, every five days, or weekly. Administration can also be continuous and adjusted to maintaining a level of the compound within any desired and specified range.

All patents, patent applications, and publications referenced herein are incorporated by reference in their entirety for all purposes.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is °C or is at ambient temperature, and pressure is at or near atmospheric. There are numerous variations and combinations of process conditions that can be used to optimize product quality and performance.

### Example 1 Preclinical Results for an Influenza Vaccine

In brief, these studies strongly suggest that: (1) GPI- cytokine-modified VLPs enhance virus-specific IgG responses in young and aged mice, (2) GPI-cytokine modified VLPs, but not VLPs alone, confer protection against heterologous influenza challenge, (3) cross-protective efficacy is granted to aged mice when immunized with GPI-cytokine modified VLPs, and (4) Aged mice demonstrate enhanced IgG responses to a heterologous virus after immunization with GPI-cytokine modified VLPs in comparison to VLP alone.

### Optimization of GPI-Cytokine Modified VLP Vaccine Production Using Protein Transfer Technology

As reported previously, the incorporation of GPI-GM-CSF is dependent on the incubation time and concentration of GPI-GM-CSF [52]. In some embodiments, the GPI-anchored polypeptide is incubated for one hour at 37° C. The incorporation was dependent on the presence of the GPI-anchor and incorporated GPI-GM-CSF was stable at least for 4 weeks [52]. VLP-Incorporated GPI-GM-CSF is also functional in activating the proliferation of dendritic cells from BALB/c mice for up to 4 weeks [52]. In addition, dual incorporation of GPI-GM-CSF and GPI-IL-12 induces maturation of both young and aged bone marrow derived dendritic cells as measured by increased expression of CD80, CD86, and MHC Class II (data not shown). These findings indicate that it is possible to produce VLPs from human viruses and modify with functionally active GPI-GM-CSF and GPI-IL-12 by protein transfer and that dendritic cells from aged mice proliferate and mature in a comparable manner to young dendritic cells to GPI-cytokine modified VLPs. In addition, we did not expect any significant differences in proliferative capacity or maturation capacity between young and aged mice [45].

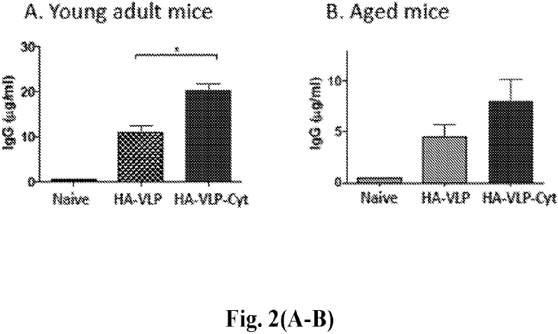

Influenza VLPs transferred with GPI-GM-CSF and GPI-IL-12 enhance homologous antibody responses in both young and aged mice (FIG. 2): GPI-cytokine influenza H1 VLPs induce enhanced total IgG antibody responses (2-fold higher than VLP) to anti-homologous H1N1 influenza A/PR8 virus in both young (FIG. 2A) and aged mice (FIG. 2B).

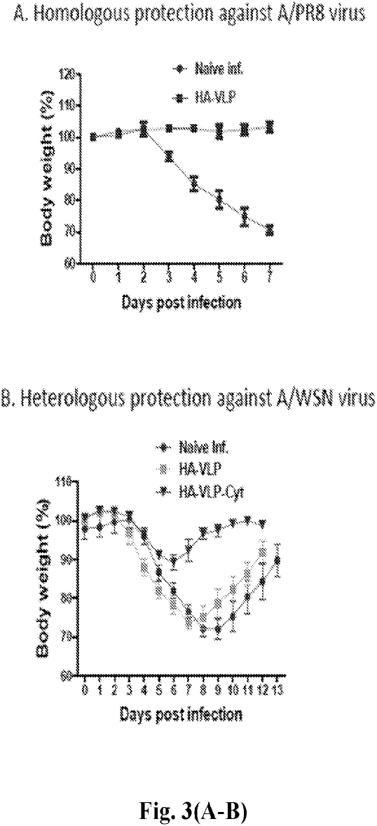

GPI-cytokine modified VLP vaccines provide enhanced cross protection against an antigenically diverse influenza virus in young adult mice (FIG. 3). Naïve or HA-VLP (PR8-derived) immunized mice were challenged with a homologous PR8 (FIG. 3A) or heterologous (FIG. 3B) Influenza (WSN strain) and weight was monitored. Although VLP only immunized mice were protected from a homologous challenge, only mice immunized with VLP-cytokine modified vaccines were protected from heterologous challenge, strongly suggesting that GPI-cytokine incorporation into VLPs enhances cross-protection against heterologous challenge.

### VLP-Based Vaccines Incorporated With GPI-Cytokines Confer Enhanced Cross Protection in Aged Mice

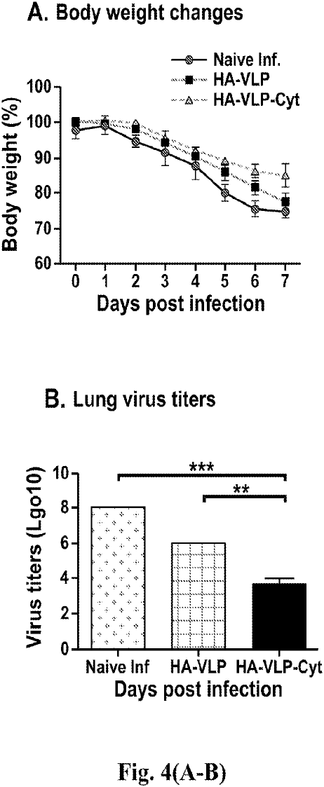

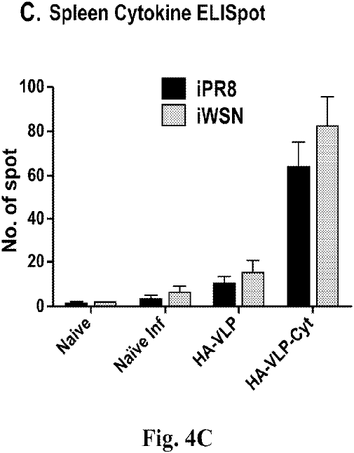

Aged mice (18 months old) were immunized as described with PBS, HA-VLP alone, or HA-VLP-Cyt. As depicted in FIG. 4A, HA-VLP-Cyt immunized mice maintained body weight more effectively and were able to better control lung virus levels after a heterologous challenge (4-log decrease compared control and 2-log compared to VLP alone, FIG. 4B). In addition, only aged mice vaccinated with HA-VLP-Cyt were able to mount strong cellular immune responses in the spleen as demonstrated by a 6-fold increase in IFN-y producing cells against both inactivated homologous (PR8) and heterologous (WSN) Influenza strains (FIG. 4C).

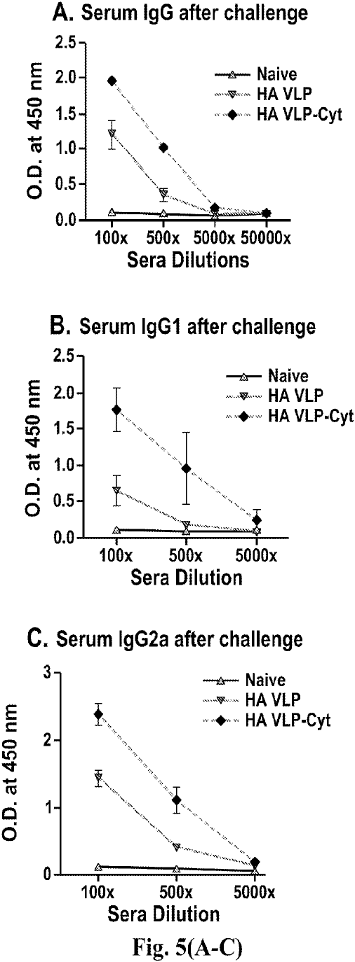

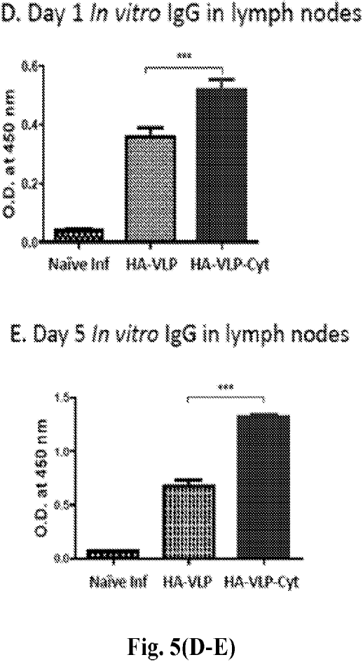

Addition of GPI-GM-CSF and GPI-IL-12 to influenza VLPs significantly enhances amnestic antibody production in aged mice. Serum antibody production against a heterologous influenza (WSN) virus was enhanced after vaccination with GPI-cytokine modified VLP in comparison to VLP alone. Total IgG, IgG1, and IgG2A responses against heterologous Influenza WSN were increased in aged mice (FIGS. 5A-C). In addition, anti-WSN antibody secreting cells from draining lymph nodes were significantly increased after vaccination with GPI-cytokine incorporated VLP in comparison to VLP alone (FIGS. 5D-E).

### Example 2. Design, Expression, and Characterization of GM-CSF-GPI S1 RBD Fusion Protein

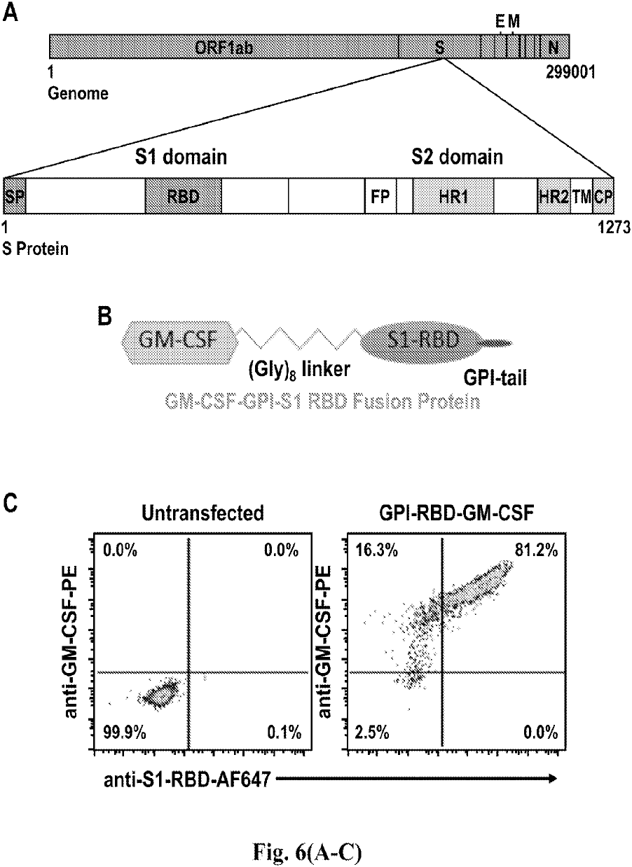

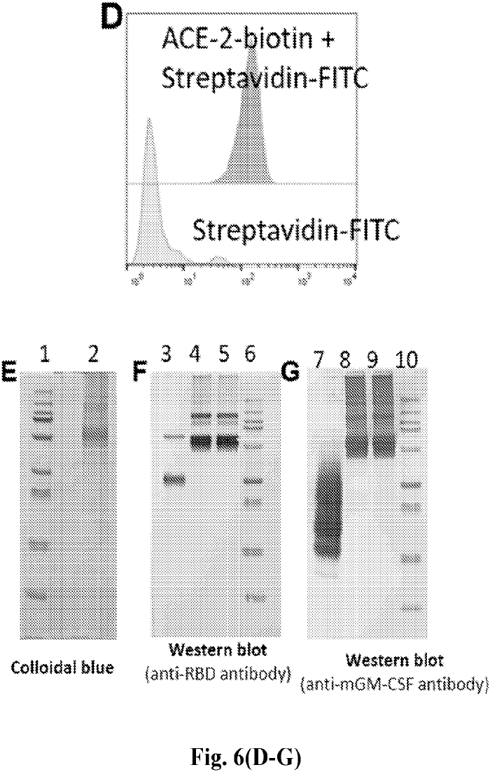

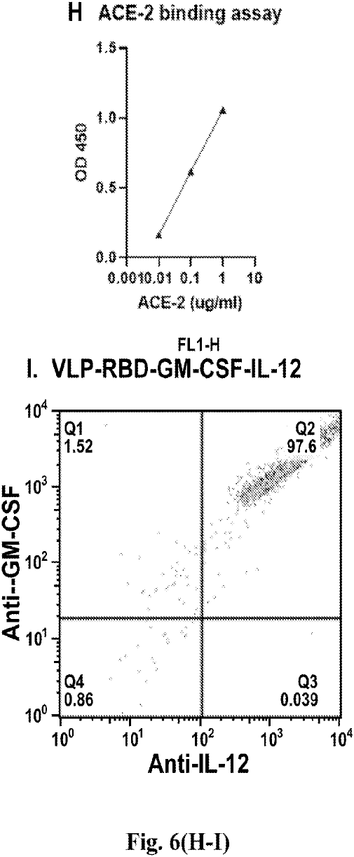

A fusion protein gene was constructed by joining the RBD domain sequence with GM-CSF and a GPI-anchor signal sequence from CD59 (FIGS. 6A and 6B). The DNA construct was then inserted into mammalian cell expression plasmid vectors and transfected into CHO-S cells. Flow cytometry was used to demonstrate dual expression of both the S1 RBD and GM-CSF on the surface of transfected CHO-S cells (FIG. 6C right dotplot). The fusion protein expressed on CHO S cells binds to its receptor human ACE2 as determined by flow cytometry (FIG. 6D).

Fusion protein was purified by Immunoaffinity chromatography using rat anti-mouse GM-CSF antibody (Clone MP1-22E9, Bio X cell) coupled to NHS-Sepharose beads. The fusion protein runs at 60-65 kDa on SDS-PAGE (FIG. 6E). while GPI-GM-CSF runs at 15-35 kDa and RBD runs 25 kDa Western blot analysis was performed for identity and size comparison of the fusion protein to wild-type mouse GPI-GM-CSF and RBD. Immunoaffinity purified fusion protein was subjected to SDS-PAGE, transferred to nitrocellulose membranes, probed with rat anti-mouse GM-CSF antibody (Clone MP1-22E9) or human anti-RBD antibody (Clone CR3022, antibodies online) and revealed with Goat anti-rat AP or Goat anti-human AP and NBT/BCIP substrate (FIGS. 6F and 6G)). Fusion protein (60-65 kDa) was detected by both anti-GM-CSF (FIG. 6F) and anti-RBD (FIG. 6G) antibodies. In addition, the ability to use affinity chromatography to purify the fusion protein with anti-GM-CSF antibody suggests that the fusion protein can be purified to a sufficient purity level for downstream applications. To determine whether the purified fusion protein retains its ability to bind ACE2, ELISA was performed and found that human ACE-2 binds to plate bound GPI-RBD-GM-CSF in a dose dependent manner (FIG. 6H). The ability of incorporation of the fusion protein into VLPs is confirmed by flow cytometry (FIG. 6I). The protein transfer process using VLPs and GPI-IL-12 and GPI-RBD-GM-CSF is depicted in the cartoon in FIG. 7.

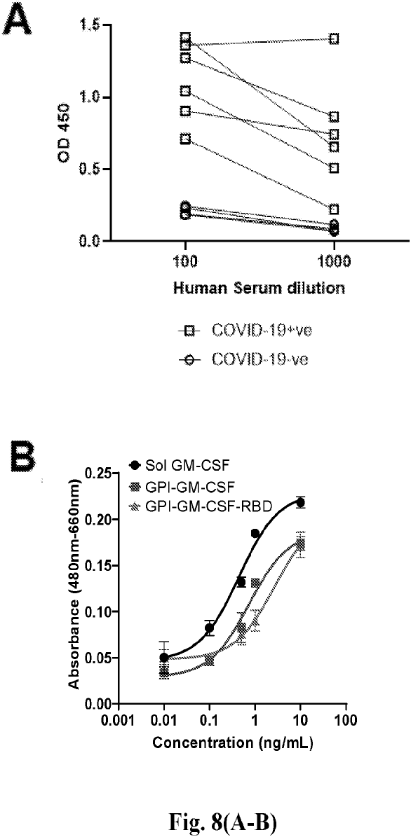

In order to determine whether GPI-RBD-GM-CSF protein can be recognized by the antibodies generated against SARS-CoV2 virus in patients, an ELISA was performed to detect the binding of antibodies in the convalescent plasma to plate bound fusion protein (FIG. 8A). To assess biological activity of GM-CSF in the fusion protein, a proliferation assay was carried out using murine bone marrow derived cells [55]. Bone marrow cells from the long bones was cultured with the GM-CSF-GPI-S1 RBD fusion protein, or recombinant soluble GM-CSF. The cell proliferation was measured by an XTT assay from Biotium (Fremont, CA) (FIG. 8B). GPI-RBD-GM-CSF fusion protein was able to induce proliferation of progenitor cells in the bone marrow on par with soluble GM-CSF, providing evidence that GM-CSF in the fusion construct has maintained biological activity.

PR8 influenza VLPs were produced as previously described [56] and as described below. Toxicology-grade Influenza VLPs (A/PR8) will be provided by Medigen, Inc. (Frederick, Maryland).

VLP Production: Virus and cells. Spodoptera frugiperda Sf9 cells were maintained in suspension in serum-free SF900II medium (GIBCO-BRL) at 27° C. in spinner flasks at a speed of 70 to 80 rpm. CV-1 and Madin-Darby canine kidney (MDCK) cells were grown and maintained in Dulbecco’s modified Eagle’s medium (DMEM). Mouse-adapted influenza A/PR8/34 and A/WSN/33 viruses were prepared as lung homogenates from intranasally infected mice. Preparation of influenza VLPs. A cDNA for influenza virus M1 (A/PR8) was obtained. The M1 gene was PCR amplified with primers containing flanking restriction enzyme sites for cloning into the pSP72 plasmid expression vector under the T7 promoter (forward primer, 5′ TCC CCCGGG CCACC ATG AGC CTT CTG ACC GAG GTC 3′ (SEQ ID NO:9); reverse primer, 5′ TTA CT TCTAGA TTA CTT GAA CCG TTG CAT CTG 3′ (SEQ ID NO:10); SmaI and XbaI sites are underlined). The pSP72 clone containing the M1 gene was confirmed by DNA sequencing, and the expression of the M1 protein was confirmed by Western blot analysis of CV-1 cells transfected with pSP72 containing the M1 gene following infection with a recombinant vaccinia virus expressing T7 polymerase. The M1 gene was subcloned into the SmaI and XbaI site in the baculovirus transfer vector pc/pS 1 containing a hybrid capsid-polyhedrin promoter. To produce a recombinant baculovirus (rBV) expressing M1, Sf9 insect cells were cotransfected with Baculogold DNA (BD/PharMingen) and the pc/pS1-M1 transfer vector by following the manufacturer’s instructions. The supernatant was harvested 5 days after transfection, and recombinant plaques expressing M1 were selected by plaque assay and expanded.

For Western blot analysis to determine the expression of M1 and HA, transfected or infected cells were dissolved in sodium dodecyl sulfate (SDS)-polyacrylamide gel electrophoresis (PAGE) sample buffer (50 mM Tris, 3% β-mercaptoethanol, 2% SDS, 10% glycerol), separated by SDS-PAGE, and then probed with mouse anti-M1 antibody (1:4,000; Serotec) and sera from PR8 virus-infected mice (1:1,000). The virus titer was determined with a Fast Plax titration kit according to the manufacturer’s instructions (Novagen, Madison, WI).

To produce VLPs containing influenza virus M1 and HA, Sf9 cells were coinfected with rBVs expressing HA and M1 at multiplicities of infection of 4 and 2, respectively. Culture supernatants were harvested at 3 days post-infection, cleared by low-speed centrifugation (2,000 × g for 20 min at 4° C.) to remove cells, and VLPs in the supernatants were pelleted by ultracentrifugation (100,000 × g for 60 min). The sedimented particles were resuspended in phosphate-buffered saline (PBS) at 4° C. overnight and further purified through a 20%-30%-60% discontinuous sucrose gradient at 100,000 × g for 1 h at 4° C. The VLP bands were collected and analyzed by using Western blots probed with anti-M1 antibody and mouse anti-PR8 sera for detecting M1 and HA, respectively. The level of residual rBV in the purified VLPs was determined by plaque assay, and equivalent titers of HA-expressing rBVs were estimated to contribute less than 5% of HA in VLPs as determined by Western blotting. The functionality of HA incorporated into VLPs was assessed by hemagglutination activity using chicken red blood cells as described previously. Alternatively, VLPs can be purified using tangential flow diafiltration followed by ion exchange chromatography (Capto Q column).

In addition, release testing assays for VLP vaccine will be established. These assays will include size of VLP as measured by a Malvern Zetasizer, overall protein concentration by micro BCA assay, incorporation efficiency of GPI-anchored cytokines and fusion protein into VLPs as measured by western blot and flow cytometry, and in vitro functional assays for biological activity of the RBD domain and GM-CSF. Binding of the RBD domain to human ACE2 receptor will be used to demonstrate functional activity. Reporter cells for human GM-CSF (Tf1, ATCC) and mouse/human IL-12 (HEK-Blue IL-12), Invivogen) are commercially available.

GPI-RBD-GM-CSF fusion protein retains structural and functional activity. To determine whether the fusion protein expressed in the CHO-S cells retain the ability to bind to the anti-RBD antibodies and retain the function of fusion partner GM-CSF, we have performed flow cytometry (FIG. 6D), ELISA (FIG. 8A) and in vitro stimulation of bone marrow derived dendritic cells (BMDC) using XTT proliferation assay (FIG. 8B). We have obtained convalescent plasma from commercial source (RayBiotech) and tested in a direct ELISA which confirmed binding of COVID-19 positive serum samples but not negative samples to the plate-bound GPI-RBD-GM-CSF fusion protein suggesting that antibodies against native spike protein from human COVID-19 patients able to bind to fusion protein (FIG. 8A). To confirm that the fusion protein retains the cytokine functional activity of GM-CSF, BMDC were cultured with various concentrations of soluble GM-CSF (BioLegend), GPI-GM-CSF and GPI-GM-CSF purified from CHO-S cells (FIG. 8B). The data suggest that GM-CSF binding to its receptor and activation of DCs is not affected by the presence of RBD.

VLP vaccine induces durable antibody response and neutralizes the virus. BALB/c (2-3 months old) mice were immunized with GPI-GM-CSF-RBD fusion protein (0.1, 1.0, 2.0 and 5.0 µg), VLPs (1.0, 2.0, 5.0 and 10 µg) incorporated with the fusion protein and GPI-IL-12 and control VLP without cytokines and commercially available RBD (Ray Biotech) or PBS. Booster dose was given 2 weeks after the first dose. The route of administration was either subcutaneous (s.c.) or intramuscular (i.m.).

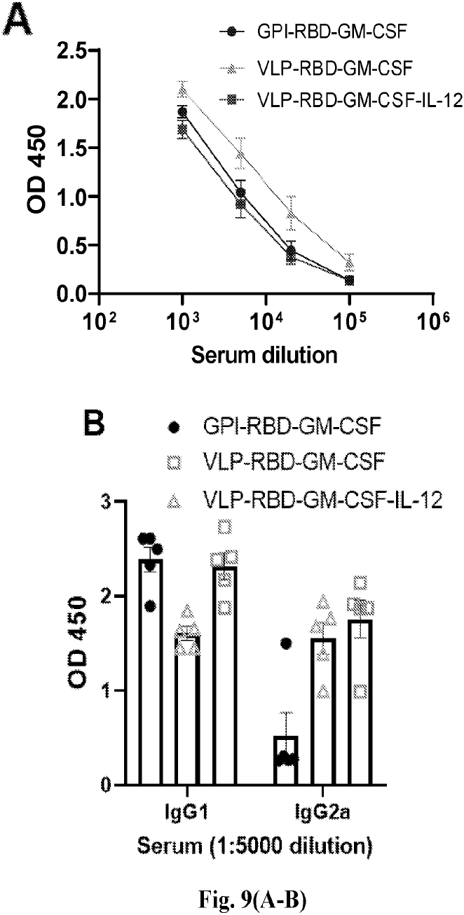

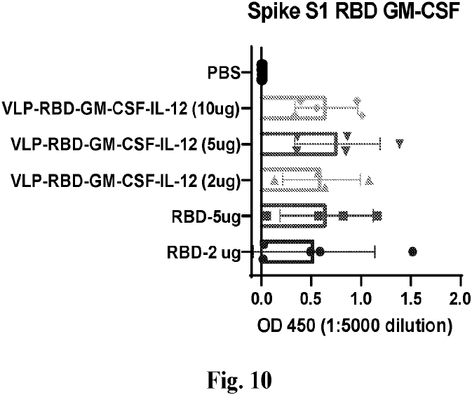

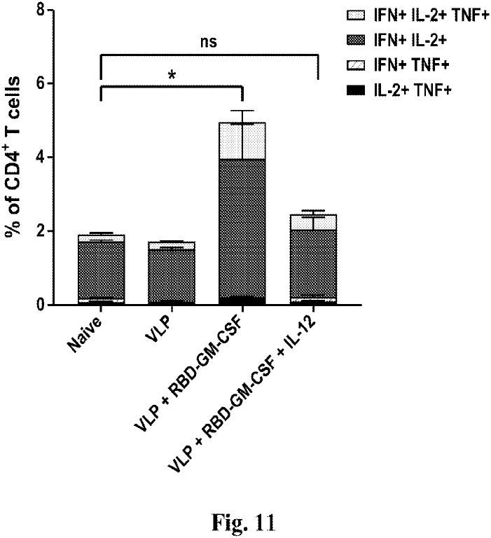

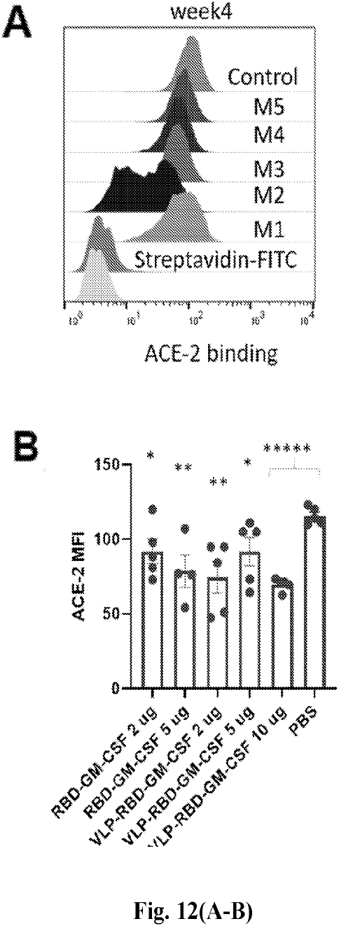

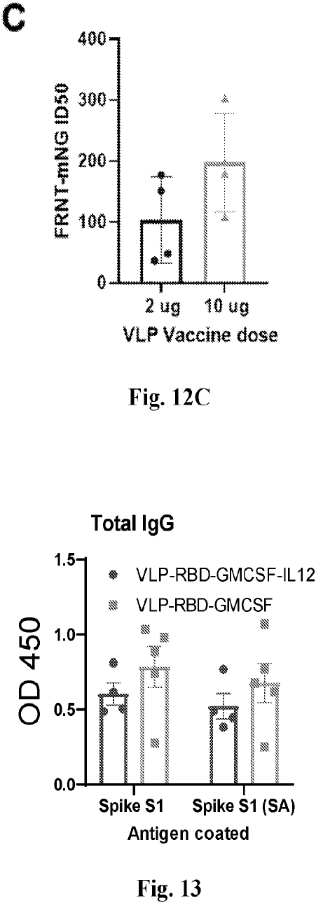

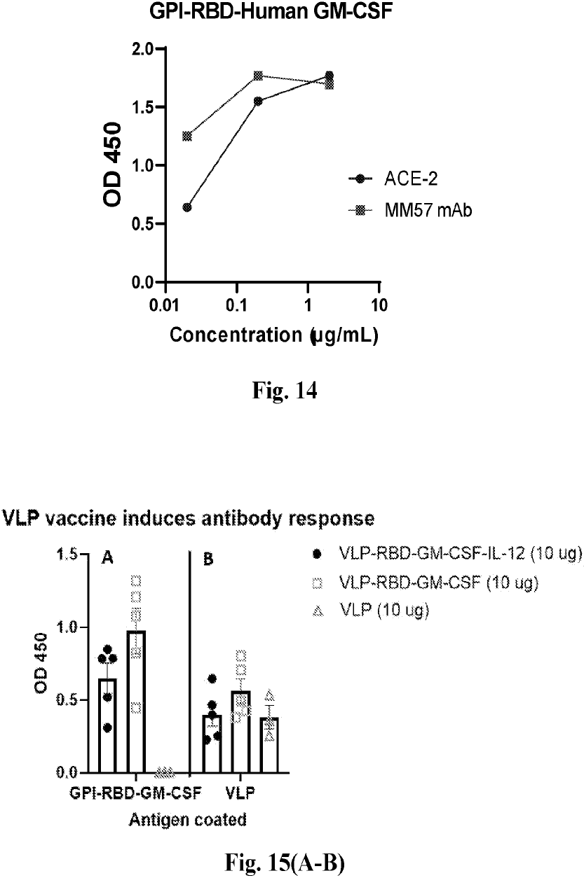

Blood was collected every 2 - 4 weeks for antibody titer, ACE2 binding inhibition, and virus microneutralization. VLP vaccine induces robust antibody response both i.m. (FIG. 9A) and s.c. (data not shown) routes. While the GPI-RBD-GM-CSF fusion protein alone induces an elevated antibody response, which is mostly Th2 type IgG1 (circles, FIG. 9B left bars), VLP vaccine with the fusion protein and GPI-IL-12 induces both IgG1 and IgG2a (a Th1-induced response; triangles and squares, FIG. 9B). Recombinant Spike RBD obtained from RayBiotech failed to induce antibody response (data not shown), suggesting that the GM-CSF in our fusion protein is acting as an adjuvant. The antibody response is durable, even after 6 months, suggesting VLP as a delivery platform is more effective than the protein alone (FIG. 10). The splenocytes response to peptide pools generated from Spike S1 RBD induced effector cytokines (Th1 cytokines IL-2, TNF-a and IFNg) suggesting that VLP vaccine with fusion protein induces not only humoral response (antibodies) but also induces T cell response (FIG. 11). Our data also suggest that the serum from VLP vaccinated mice blocks binding of fusion protein to ACE2 (FIG. 12A & B) and inhibits infection of WA1 strain of SARS-CoV-2 in vitro using Vero E6 cells (FIG. 12C). In order to determine whether the antibodies induced by fusion protein bind to the new variant observed in South Africa (SA variant B1. 351), we have performed an ELISA by coating the plate with either original S1 or SA variant S1 (Sino Biologicals) and obesrved that the mouse sera from VLP vaccine with fusion protein bind equally well to SA variant (FIG. 13). To furthe confirm whether the GPI-RBD fusion protein with human GM-CSF binds to human ACE2, an ELISA was performed and confirmed that replacing mouse GM-CSF with human GM-CSF in the fusion protein does not affect it’s bind to ACE-2 (FIG. 14). To confirm whether the VLP vaccine with fusion protein induces antibodies against VLP antigens inaddition to S1 RBD, we have performed an ELISA by coating the plate with the fusion protein (FIG. 15A) or VLP (FIG. 15B) and observed that mice immunized with VLP vaccine with fusion protein produce antibodies against VLP vantigens and S1 RBD. Our results are consistent with the recent report by the Bjorkman laboratory (62) demonstrating the requirement of VLP as a delivery vehicle for antigens.

Publications cited herein are hereby specifically incorporated by reference in their entireties and at least for the material for which they are cited.

### REFERENCES

1. Zhou P, Yang XL, Wang XG, Hu B, Zhang L, Zhang W, Si HR, Zhu Y, Li B, Huang CL, Chen HD, Chen J, Luo Y, Guo H, Jiang RD, Liu MQ, Chen Y, Shen XR, Wang X, Zheng XS, Zhao K, Chen QJ, Deng F, Liu LL, Yan B, Zhan FX, Wang YY, Xiao GF, Shi ZL. 2020. A pneumonia outbreak associated with a new coronavirus of probable bat origin. Nature 579: 270-3
2. Lan J, Ge J, Yu J, Shan S, Zhou H, Fan S, Zhang Q, Shi X, Wang Q, Zhang L, Wang X. 2020. Structure of the SARS-CoV-2 spike receptor-binding domain bound to the ACE2 receptor. Nature
3. Yong CY, Ong HK, Yeap SK, Ho KL, Tan WS. 2019. Recent Advances in the Vaccine Development Against Middle East Respiratory Syndrome-Coronavirus. Front Microbiol 10: 1781
4. Cao X. 2020. COVID-19: immunopathology and its implications for therapy. Nat Rev Immunol
5. Tetro JA. 2020. Is COVID-19 receiving ADE from other coronaviruses? Microbes Infect 22: 72-3
6. Peeples L. 2020. News Feature: Avoiding pitfalls in the pursuit of a COVID-19 vaccine. Proc Natl Acad Sci U S A 117: 8218-21
7. Amanat F, Krammer F. 2020. SARS-CoV-2 Vaccines: Status Report. Immunity 52: 583-9
8. Okba NM, Raj VS, Haagmans BL. 2017. Middle East respiratory syndrome coronavirus vaccines: current status and novel approaches. Curr Opin Virol 23: 49-58
9. Du L, Tai W, Yang Y, Zhao G, Zhu Q, Sun S, Liu C, Tao X, Tseng CK, Perlman S, Jiang S, Zhou Y, Li F. 2016. Introduction of neutralizing immunogenicity index to the rational design of MERS coronavirus subunit vaccines. Nat Commun 7: 13473
10. Yuan M, Wu NC, Zhu X, Lee CD, So RTY, Lv H, Mok CKP, Wilson IA. 2020. A highly conserved cryptic epitope in the receptor-binding domains of SARS-CoV-2 and SARS-CoV. Science
11. Tian X, Li C, Huang A, Xia S, Lu S, Shi Z, Lu L, Jiang S, Yang Z, Wu Y, Ying T. 2020. Potent binding of 2019 novel coronavirus spike protein by a SARS coronavirus-specific human monoclonal antibody. Emerg Microbes Infect 9: 382-5
12. Quinlan BD, Mou H, Zhang L, Guo Y, He W, Ojha A, Parcells MS, Luo G, Li W, Zhong G, Choe H, Farzan M. 2020. The SARS-CoV-2 receptor-binding domain elicits a potent neutralizing response without antibody-dependent enhancement. bioRxiv: 2020.04.10.036418
13. McElhaney JE, Kuchel GA, Zhou X, Swain SL, Haynes L. 2016. T-Cell Immunity to Influenza in Older Adults: A Pathophysiological Framework for Development of More Effective Vaccines. Front Immunol 7: 41
14. Nikolich-Zugich J. 2014. Aging of the T cell compartment in mice and humans: from no naive expectations to foggy memories. J Immunol 193: 2622-9
15. Haq K, McElhaney JE. 2014. Immunosenescence: Influenza vaccination and the elderly. Curr Opin Immunol 29: 38-42
16. Reed SG, Orr MT, Fox CB. 2013. Key roles of adjuvants in modern vaccines. Nat Med 19: 1597-608
17. Black S, De Gregorio E, Rappuoli R. 2015. Developing vaccines for an aging population. Sci Transl Med 7: 281ps8
18. Van Buynder PG, Konrad S, Van Buynder JL, Brodkin E, Krajden M, Ramler G, Bigham M. 2013. The comparative effectiveness of adjuvanted and unadjuvanted trivalent inactivated influenza vaccine (TIV) in the elderly. Vaccine 31: 6122-8
19. Mannino S, Villa M, Apolone G, Weiss NS, Groth N, Aquino I, Boldori L, Caramaschi F, Gattinoni A, Malchiodi G, Rothman KJ. 2012. Effectiveness of adjuvanted influenza vaccination in elderly subjects in northern Italy. Am J Epidemiol 176: 527-33
20. Rumke HC, Richardus JH, Rombo L, Pauksens K, Plassmann G, Durand C, Devaster JM, Dewe W, Oostvogels L. 2013. Selection of an adjuvant for seasonal influenza vaccine in elderly people: modelling immunogenicity from a randomized trial. BMC Infect Dis 13: 348
21. McElhaney JE, Beran J, Devaster JM, Esen M, Launay O, Leroux-Roels G, Ruiz-Palacios GM, van Essen GA, Caplanusi A, Claeys C, Durand C, Duval X, El Idrissi M, Falsey AR, Feldman G, Frey SE, Galtier F, Hwang SJ, Innis BL, Kovac M, Kremsner P, McNeil S, Nowakowski A, Richardus JH, Trofa A, Oostvogels L, Influence65 study g. 2013. AS03-adjuvanted versus non-adjuvanted inactivated trivalent influenza vaccine against seasonal influenza in elderly people: a phase 3 randomised trial. Lancet Infect Dis 13: 485-96
22. Boraschi D, Italiani P. 2014. Immunosenescence and vaccine failure in the elderly: strategies for improving response. Immunol Lett 162: 346-53
23. Ramirez A, Co M, Mathew A. 2016. CpG Improves Influenza Vaccine Efficacy in Young Adult but Not Aged Mice. PLoS One 11: e0150425
24. Chougnet CA, Thacker RI, Shehata HM, Hennies CM, Lehn MA, Lages CS, Janssen EM. 2015. Loss of Phagocytic and Antigen Cross-Presenting Capacity in Aging Dendritic Cells Is Associated with Mitochondrial Dysfunction. J Immunol 195: 2624-32
25. Gupta RK. 1998. Aluminum compounds as vaccine adjuvants. Adv Drug Deliv Rev 32: 155-72
26. Relyveld EH, Bizzini B, Gupta RK. 1998. Rational approaches to reduce adverse reactions in man to vaccines containing tetanus and diphtheria toxoids. Vaccine 16: 1016-23
27. Quan FS, Ko EJ, Kwon YM, Joo KH, Compans RW, Kang SM. 2013. Mucosal adjuvants for influenza virus-like particle vaccine. Viral Immunol 26: 385-95
28. Goronzy JJ, Weyand CM. 2013. Understanding immunosenescence to improve responses to vaccines. Nat Immunol 14: 428-36
29. Zhang W, Brahmakshatriya V, Swain SL. 2014. CD4 T cell defects in the aged: causes, consequences and strategies to circumvent. Exp Gerontol 54: 67-70
30. Jaffee EM. 1999. Immunotherapy of cancer. Ann N Y Acad Sci 886: 67-72
31. Trinchieri G, Scott P. 1999. Interleukin-12: basic principles and clinical applications. Curr Top Microbiol Immunol 238: 57-78
32. Vagliani M, Rodolfo M, Cavallo F, Parenza M, Melani C, Parmiani G, Forni G, Colombo MP. 1996. Interleukin 12 potentiates the curative effect of a vaccine based on interleukin 2-transduced tumor cells. Cancer Res 56: 467-70
33. Trinchieri G. 1994. Interleukin-12: a cytokine produced by antigen-presenting cells with immunoregulatory functions in the generation of T-helper cells type 1 and cytotoxic lymphocytes. Blood 84: 4008-27
34. Car BD, Eng VM, Lipman JM, Anderson TD. 1999. The toxicology of interleukin-12: a review. Toxicol Pathol 27: 58-63
35. Bajetta E, Del Vecchio M, Mortarini R, Nadeau R, Rakhit A, Rimassa L, Fowst C, Borri A, Anichini A, Parmiani G. 1998. Pilot study of subcutaneous recombinant human interleukin 12 in metastatic melanoma. Clin Cancer Res 4: 75-85
36. Brunda MJ, Luistro L, Rumennik L, Wright RB, Dvorozniak M, Aglione A, Wigginton JM, Wiltrout RH, Hendrzak JA, Palleroni AV. 1996. Antitumor activity of interleukin 12 in preclinical models. Cancer Chemother Pharmacol 38 Suppl: S16-21
37. Chen L, Chen D, Block E, O′Donnell M, Kufe DW, Clinton SK. 1997. Eradication of murine bladder carcinoma by intratumor injection of a bicistronic adenoviral vector carrying cDNAs for the IL-12 heterodimer and its inhibition by the IL-12 p40 subunit homodimer. J Immunol 159: 351-9
38. Tsung K, Meko JB, Tsung YL, Peplinski GR, Norton JA. 1998. Immune response against large tumors eradicated by treatment with cyclophosphamide and IL-12. J Immunol 160: 1369-77
39. Cohen J. 1995. IL-12 deaths: explanation and a puzzle. Science 270: 908
40. Villarino A, Hibbert L, Lieberman L, Wilson E, Mak T, Yoshida H, Kastelein RA, Saris C, Hunter CA. 2003. The IL-27R (WSX-1) is required to suppress T cell hyperactivity during infection. Immunity 19: 645-55
41. Ou-Yang P, Hwang LH, Tao MH, Chiang BL, Chen DS. 2002. Co-delivery of GM-CSF gene enhances the immune responses of hepatitis C viral core protein-expressing DNA vaccine: role of dendritic cells. J Med Virol 66: 320-8
42. Mach N, Gillessen S, Wilson SB, Sheehan C, Mihm M, Dranoff G. 2000. Differences in dendritic cells stimulated in vivo by tumors engineered to secrete granulocyte-macrophage colony-stimulating factor or Flt3-ligand. Cancer Res 60: 3239-46
43. Poloso NJ, Nagarajan S, Mejia-Oneta JM, Selvaraj P. 2002. GPI-anchoring of GM-CSF results in active membrane-bound and partially shed cytokine. Mol Immunol 38: 803-16
44. Skountzou I, Quan FS, Gangadhara S, Ye L, Vzorov A, Selvaraj P, Jacob J, Compans RW, Kang SM. 2007. Incorporation of glycosylphosphatidylinositol-anchored granulocyte-macrophage colony-stimulating factor or CD40 ligand enhances immunogenicity of chimeric simian immunodeficiency virus-like particles. J Virol 81: 1083-94
45. Lung TL, Saurwein-Teissl M, Parson W, Schonitzer D, Grubeck-Loebenstein B. 2000. Unimpaired dendritic cells can be derived from monocytes in old age and can mobilize residual function in senescent T cells. Vaccine 18: 1606-12
46. Kantoff PW, Higano CS, Shore ND, Berger ER, Small EJ, Penson DF, Redfern CH, Ferrari AC, Dreicer R, Sims RB, Xu Y, Frohlich MW, Schellhammer PF, Investigators IS. 2010. Sipuleucel-T immunotherapy for castration-resistant prostate cancer. N Engl J Med 363: 411-22
47. Higano CS, Schellhammer PF, Small EJ, Burch PA, Nemunaitis J, Yuh L, Provost N, Frohlich MW. 2009. Integrated data from 2 randomized, double-blind, placebo-controlled, phase 3 trials of active cellular immunotherapy with sipuleucel-T in advanced prostate cancer. Cancer 115: 3670-9
48. Slingluff CL, Jr., Petroni GR, Olson WC, Smolkin ME, Ross MI, Haas NB, Grosh WW, Boisvert ME, Kirkwood JM, Chianese-Bullock KA. 2009. Effect of granulocyte/macrophage colony-stimulating factor on circulating CD8+ and CD4+ T-cell responses to a multipeptide melanoma vaccine: outcome of a multicenter randomized trial. Clin Cancer Res 15: 7036-44
49. Faries MB, Hsueh EC, Ye X, Hoban M, Morton DL. 2009. Effect of granulocyte/macrophage colony-stimulating factor on vaccination with an allogeneic whole-cell melanoma vaccine. Clin Cancer Res 15: 7029-35
50. Scheibenbogen C, Schadendorf D, Bechrakis NE, Nagorsen D, Hofmann U, Servetopoulou F, Letsch A, Philipp A, Foerster MH, Schmittel A, Thiel E, Keilholz U. 2003. Effects of granulocyte-macrophage colony-stimulating factor and foreign helper protein as immunologic adjuvants on the T-cell response to vaccination with tyrosinase peptides. Int J Cancer 104: 188-94
51. Kirkwood JM, Lee S, Moschos SJ, Albertini MR, Michalak JC, Sander C, Whiteside T, Butterfield LH, Weiner L. 2009. Immunogenicity and antitumor effects of vaccination with peptide vaccine+/-granulocyte-monocyte colony-stimulating factor and/or IFN-alpha2b in advanced metastatic melanoma: Eastern Cooperative Oncology Group Phase II Trial E1696. Clin Cancer Res 15: 1443-51
52. Patel JM, Kim MC, Vartabedian VF, Lee YN, He S, Song JM, Choi HJ, Yamanaka S, Amaram N, Lukacher A, Montemagno CD, Compans RW, Kang SM, Selvaraj P. 2015. Protein transfer-mediated surface engineering to adjuvantate virus-like nanoparticles for enhanced anti-viral immune responses. Nanomedicine 11: 1097-107
53. Patel J, He S, Amaram N, Yamanaka S, Vartabedian V, Radhakrishnan V, Song JM, Shashidharamurthy R, Compans R, Kang S-m, Selvaraj P. 2013. GPI-GM-CSF protein transferred onto H5 influenza VLPs remains stably expressed and functionally active (P6168). The Journal of Immunology 190: 66.17-66.17
54. Wang BZ, Quan FS, Kang SM, Bozja J, Skountzou I, Compans RW. 2008. Incorporation of membrane-anchored flagellin into influenza virus-like particles enhances the breadth of immune responses. J Virol 82: 11813-23
55. Inaba K, Inaba M Fau - Romani N, Romani N Fau - Aya H, Aya H Fau - Deguchi M, Deguchi M Fau - Ikehara S, Ikehara S Fau - Muramatsu S, Muramatsu S Fau - Steinman RM, Steinman RM. Generation of large numbers of dendritic cells from mouse bone marrow cultures supplemented with granulocyte/macrophage colony-stimulating factor.
56. Quan FS, Huang C, Compans RW, Kang SM. 2007. Virus-like particle vaccine induces protective immunity against homologous and heterologous strains of influenza virus. J Virol 81: 3514-24
57. Zhi Y, Kobinger GP, Jordan H, Suchma K, Weiss SR, Shen H, Schumer G, Gao G, Boyer JL, Crystal RG, Wilson JM. 2005. Identification of murine CD8 T cell epitopes in codon-optimized SARS-associated coronavirus spike protein. Virology 335: 34-45
58. Moore BJB, June CH. 2020. Cytokine release syndrome in severe COVID-19. Science
59. McCray PB, Jr., Pewe L Fau - Wohlford-Lenane C, Wohlford-Lenane C Fau - Hickey M, Hickey M Fau - Manzel L, Manzel L Fau - Shi L, Shi L Fau - Netland J, Netland J Fau -Jia HP, Jia Hp Fau - Halabi C, Halabi C Fau - Sigmund CD, Sigmund Cd Fau - Meyerholz DK, Meyerholz Dk Fau - Kirby P, Kirby P Fau - Look DC, Look Dc Fau - Perlman S, Perlman S. Lethal infection of K18-hACE2 mice infected with severe acute respiratory syndrome coronavirus.
60. Bao L, Deng W, Huang B, Gao H, Liu J, Ren L, Wei Q, Yu P, Xu Y, Qi F, Qu Y, Li F, Lv Q, Wang W, Xue J, Gong S, Liu M, Wang G, Wang S, Song Z, Zhao L, Liu P, Zhao L, Ye F, Wang H, Zhou W, Zhu N, Zhen W, Yu H, Zhang X, Guo L, Chen L, Wang C, Wang Y, Wang X, Xiao Y, Sun Q, Liu H, Zhu F, Ma C, Yan L, Yang M, Han J, Xu W, Tan W, Peng X, Jin Q, Wu G, Qin C. 2020. The Pathogenicity of SARS-CoV-2 in hACE2 Transgenic Mice. bioRxiv: 2020.02.07.939389
61. Cohen J. 2020. From mice to monkeys, animals studied for coronavirus answers. Science 368: 221
62. Chan JF-W, Zhang AJ, Yuan S, Poon VK-M, Chan CC-S, Lee AC-Y, Chan W-M, Fan Z, Tsoi H-W, Wen L, Liang R, Cao J, Chen Y, Tang K, Luo C, Cai J-P, Kok K-H, Chu H, Chan K-H, Sridhar S, Chen Z, Chen H, To KK-W, Yuen K-Y. 2020. Simulation of the clinical and pathological manifestations of Coronavirus Disease 2019 (COVID-19) in golden Syrian hamster model: implications for disease pathogenesis and transmissibility. Clinical Infectious Diseases
63. Sia SFY, Li-Meng; Chin, Alex WH; Fung, Kevin; Poon, Leo LM; Nicholls, John M.; Peiris, Malik; Yen, Hui-Ling. 2020. Pathogenesis and transmission of SARS-CoV-2 virus in golden Syrian hamsters. PREPRINT (Version 1), available at Resesearch Square 01 Apr. 2020
64. Nemunaitis J. Vaccines in cancer: GVAX, a GM-CSF gene vaccine.
65. Borrello I, Pardoll D. GM-CSF-based cellular vaccines: a review of the clinical experience.
66. Pan WY, Lo Ch Fau - Chen C-C, Chen Cc Fau - Wu P-Y, Wu Py Fau - Roffler SR, Roffler Sr Fau - Shyue S-K, Shyue Sk Fau - Tao M-H, Tao MH. Cancer immunotherapy using a membrane-bound interleukin-12 with B7-1 transmembrane and cytoplasmic domains.
67. Nagarajan S, Selvaraj P. Glycolipid-anchored IL-12 expressed on tumor cell surface induces antitumor immune response.
68. Atkins MB, Robertson Mj Fau - Gordon M, Gordon M Fau - Lotze MT, Lotze Mt Fau -DeCoste M, DeCoste M Fau - DuBois JS, DuBois Js Fau - Ritz J, Ritz J Fau - Sandler AB, Sandler Ab Fau - Edington HD, Edington Hd Fau - Garzone PD, Garzone Pd Fau - Mier JW, Mier Jw Fau - Canning CM, Canning Cm Fau - Battiato L, Battiato L Fau - Tahara H, Tahara H Fau - Sherman ML, Sherman ML. Phase I evaluation of intravenous recombinant human interleukin 12 in patients with advanced malignancies.
69. Zhou X, Hopkins JW, Wang C, Brahmakshatriya V, Swain SL, Kuchel GA, Haynes L, McElhaney JE. 2016. IL-2 and IL-6 cooperate to enhance the generation of influenza-specific CD8 T cells responding to live influenza virus in aged mice and humans. Oncotarget 7: 39171-83
70. 2002. UN Population Division: World population Ageing 1950-2050. New York: United Nations

What is claimed is:

1. An isolated recombinant polypeptide comprising a SARS-CoV-2 S1 protein binding domain polypeptide and a GM-CSF polypeptide.

2. The recombinant polypeptide of claim 1, wherein the SARS-CoV-2 S1 protein binding domain polypeptide comprises a full-length S1 polypeptide.

3. The recombinant polypeptide of claim 1 or claim 2, wherein the SARS-CoV-2 S1 protein binding domain polypeptide comprises SEQ ID NO: 1.

4. The recombinant polypeptide of claim 1 or claim 2, wherein the SARS-CoV-2 S1 protein binding domain polypeptide comprises a sequence at least 80% identical to SEQ ID NO:1.

5. The recombinant polypeptide of claim 1 or claim 2, wherein the SARS-CoV-2 S1 protein binding domain polypeptide comprises SEQ ID NO:6 or SEQ ID NO:7.

6. The recombinant polypeptide of claim 1 or claim 2, wherein the SARS-CoV-2 S1 protein binding domain polypeptide comprises a sequence at least 80% identical to SEQ ID NO:6 or SEQ ID NO:7.

7. The recombinant polypeptide of any one of claims 1-6, wherein the GM-CSF polypeptide comprises SEQ ID NO:2.

8. The recombinant polypeptide of any one of claims 1-6, wherein the GM-CSF polypeptide is at least 80% identical to SEQ ID NO:2.

9. An isolated recombinant polynucleotide comprising a SARS-CoV-2 S1 protein receptor binding domain polynucleotide, a GM-CSF polynucleotide and a GPI-anchoring signal polynucleotide.

10. The recombinant polynucleotide of claim 9, wherein the SARS-CoV-2 S1 protein receptor binding domain polynucleotide and the GM-CSF polynucleotide are operably linked.

11. The recombinant polynucleotide of claim 9, wherein the SARS-CoV-2 S1 protein receptor binding domain polynucleotide comprises a full-length S1 polynucleotide.

12. The recombinant polynucleotide of claim 9, wherein the recombinant polynucleotide comprises SEQ ID NO:12 or SEQ ID NO:14.

13. A method of detecting an antibody specific for a SARS-CoV-2 S1 protein receptor binding domain in a subject comprising, obtaining a blood sample from the subject, contacting the sample with a recombinant polypeptide comprising a SARS-CoV-2 S1 protein receptor binding domain polypeptide and a GM-CSF polypeptide, and detecting specific binding between the recombinant polypeptide and an antibody in the blood sample, wherein detection of specific binding indicates a presence of the antibody specific for a SARS-CoV-2 S1 protein receptor binding domain in the subject.

14. The method of claim 13, further comprising treating the subject for a SARS-CoV-2 infection.

15. The method of claim 13 or claim 14, wherein the subject is a human.

16. An enveloped virus-like particle (VLP) comprising a GPI-recombinant polypeptide anchored to the VLP, wherein the GPI-recombinant polypeptide comprises a SARS-CoV-2 S1 protein receptor binding domain polypeptide and a GM-CSF polypeptide (GPI-RBD-GM-CSF polypeptide).

17. The enveloped VLP of claim 16, further comprising one or more GPI-immunostimulatory molecules anchored to the VLP.

18. The enveloped VLP of claim 17, wherein the one or more GPI-immunostimulatory molecules is a GPI-IL-12 molecule.

19. The enveloped VLP of any one of claims 16-18, wherein the VLP is derived from an influenza virus.

20. The enveloped VLP of any one of claims 16-19, wherein the VLP is derived from an influenza virus A/PR8.

21. The enveloped VLP of any one of claims 16-20, wherein the SARS-CoV-2 S1 protein receptor binding domain polypeptide comprises SEQ ID NO:1.

22. The enveloped VLP of any one of claims 16-20, wherein the SARS-CoV-2 S1 protein binding domain polypeptide comprises a sequence at least 80% identical to SEQ ID NO:1.

23. The enveloped VLP of any one of claims 16-22, wherein the GM-CSF polypeptide comprises SEQ ID NO:2.

24. The enveloped VLP of any one of claims 16-22, wherein the GM-CSF polypeptide is at least 80% identical to SEQ ID NO:2.

25. A method for reducing a SARS-CoV-2 infection in a subject, comprising administering to the subject a therapeutically effective amount of an enveloped virus-like particle (VLP) of any of claims 16-24, wherein the infection in the subject is reduced as compared to a control.

26. The method of claim 25, wherein the enveloped VLP further comprises one or more GPI-immunostimulatory molecules anchored to the VLP.

27. The method of claim 26, wherein the one or more GPI-immunostimulatory molecules is a GPI-IL-12 molecule.


SEQUENCE LISTING (14 sequences)

SEQ ID NO: 1 - protein, 223 aa, Artificial Sequence
  Synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 2 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 3 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 27 aa, Artificial Sequence
  Synthetic construct
NGGTSLSEKTVLLLVTPFLAAAWSLHP

SEQ ID NO: 6 - protein, 1213 aa, Artificial Sequence
  SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 7 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - DNA, 3822 bp, Artificial Sequence
  Synthetic construct
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 9 - DNA, 35 bp, Artificial Sequence
  Synthetic construct
tcccccgggccaccatgagccttctgaccgaggtc

SEQ ID NO: 10 - DNA, 32 bp, Artificial Sequence
  Synthetic construct
ttacttctagattacttgaaccgttgcatctg

SEQ ID NO: 11 - DNA, 1200 bp, Artificial Sequence
  Synthetic construct
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaaggaggaggaggaggaggaggaggaagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaatggtgggacatccttatcagagaaaacagttcttctgctggtgactccatttctggcagcagcctggagccttcatccctaa

SEQ ID NO: 12 - DNA, 1209 bp, Artificial Sequence
  Synthetic construct
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggaggaggaggaggaggaggaggaagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaatggtgggacatccttatcagagaaaacagttcttctgctggtgactccatttctggcagcagcctggagccttcatccctaa

SEQ ID NO: 13 - DNA, 1230 bp, Artificial Sequence
  Synthetic construct
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaaggaggaggaggaggaggaggaggaagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttaatggtgggacatccttatcagagaaaacagttcttctgctggtgactccatttctggcagcagcctggagccttcatccctaa

SEQ ID NO: 14 - DNA, 1209 bp, Artificial Sequence
  Synthetic construct
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggaggaggaggaggaggaggaggaagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaatggtgggacatccttatcagagaaaacagttcttctgctggtgactccatttctggcagcagcctggagccttcatccctaa